(19) United States
(12) Patent Application Publication
(10) Pub. No.: US 2016/0244482 A1
(43) Pub. Date: Aug. 25, 2016

(54) Method of Extracting Protein

(71) Applicant: ELASTAGEN PTY LTD, Eveleigh, New South Wales (AU)

(72) Inventor: Phillip Elliott, Thebarton (AU)

(21) Appl. No.: 15/024,253

(22) PCT Filed: Sep. 24, 2014
(86) PCT No.: PCT/AU2014/000932

§ 371 Date: Mar. 24, 2016

(30) Foreign Application Priority Data
Sep. 24, 2013 (AU) 2013903669

(51) Int. Cl. C07K 1/14

(52) U.S. Cl. CPC C07K 1/145; C07K 2319/00; A61K 38/00

(57) ABSTRACT

The present disclosure provides a method for extracting a soluble protein from a population of microorganisms, the method comprising contacting the population of microorganisms expressing the soluble protein with an amount of a solution comprising from about 1% (v/v) to less than 50% (v/v) carboxylic acid effective to extract the soluble protein from the population of microorganisms.

### RELATED APPLICATION DATA

The present applications claims priority from Australian Provisional Patent Application No. 2013903669 entitled “Method of extracting protein” filed on 24 Sep. 2013. The entire contents of that application are hereby incorporated by reference.

### FIELD

The present disclosure relates to methods for extracting proteins from microorganisms.

### BACKGROUND

Microorganisms permit production of recombinant proteins at high levels in relatively short periods of time. Moreover, many microorganisms can be genetically modified relatively easily to express a protein of interest at a high level. Accordingly, microorganisms are one of the cell types of choice for production of proteins for industrial application, e.g., for therapeutic or diagnostic or cosmetic applications.

For many applications, proteins expressed in microorganisms must be extracted and purified to at least some extent. To purify these proteins it is often necessary to lyse the cell using methods known in the art such as homogenization or chemical lysis. However, releasing proteins by these methods also results in the release of a large number of additional proteins, as well as nucleic acids, lipids, endotoxins and other cell components from the microorganism. This is particularly the case when purifying proteins that are soluble within a microorganism, as opposed to proteins included in aggregates or inclusion bodies, since many of the contaminants are also soluble within the microorganism. These contaminating proteins and other substances are often extracted with the protein of interest. Accordingly, the protein of interest must then be purified to remove these contaminants. Higher concentrations of contaminants can require more purification steps and/or more reagents, resulting in increased cost to produce a purified protein.

Traditional protein extraction methods also often make use of organic solvents that are expensive and are often highly flammable, thus requiring specialized equipment for containment, fireproofing, explosion proofing and disposal. Accordingly, it is desirable not to use such solvents, particularly when purifying large amounts of protein.

The use of harsh organic solvents can also require specialized equipment that does not degrade or corrode during the extraction process. Alternatively, the equipment used in the extraction method must be replaced regularly due to degradation/corrosion.

Some methods for extracting protein from microorganisms also make use of enzymes, such as lysozyme. However, these methods are difficult to use to extract proteins from large amount of cells because the lysozyme addition is inefficient and it is difficult to disperse the enzyme throughout a large pellet of cells.

It will be apparent to the skilled artisan from the foregoing that methods that permit extraction of a protein from a microorganism and reduce the level of contamination by nucleic acids and/or lipids and/or endotoxins and/or other cell proteins from the cell are desirable. Desirably, the method permits the protein to be extracted in soluble form, while contaminants are precipitated.

### SUMMARY

The present disclosure is based on the inventor's development of a method that enabled extraction of proteins, e.g., soluble proteins from microorganisms, in which the proteins remained soluble while a considerable amount of the endogenous proteins, nucleic acid and/or lipids from the microorganism precipitated. The method enabled the inventor to extract the soluble proteins at a higher purity than some other methods, while extracting similar levels of protein to those methods. Thus, the method produced by the inventor facilitates downstream purification steps by recovering a high yield of protein with a reduced level of contamination. As exemplified herein, the inventor has used multiple carboxylic acids to extract soluble proteins from bacteria, e.g., Escherichia coli. These results are used as a model for microorganisms generally.

In one example, the inventor has extracted soluble protein at a high level of purity using a sufficiently low concentration of a carboxylic acid (e.g., less than about 50% of a carboxylic acid) that the method could be performed with standard equipment. Some examples of the method produced by the inventor permit the extracted protein to be applied directly to a filter, e.g., a filtration membrane commonly used in tangential flow filtration or depth filtration without the need for adding a buffer to reduce the risk of damaging the filter. The inventor also found that increasing concentrations of a carboxylic acid (e.g., acetic acid) above about 37% or 50% or 62% reduced the purity of the protein, e.g., to a level less than that when the protein was extracted with about 25% of the acid. In this regard, using a solution comprising about 25% of a carboxylic acid resulted in a high level of purity. Extraction using a solution comprising carboxylic acid at 100% resulted in sufficiently pure protein for further purification, however the protein was not as pure as the protein extracted with a solution comprising a carboxylic acid at about 25%.

In another example, the inventor found that extracting the protein using between about 25% to about 100% of a carboxylic acid for more than about, e.g., 20 minutes or 30 minutes had little effect on the amount of protein extracted. Thus, the inventor has determined that extraction can be performed in a relatively short period of time (if desired).

As will be apparent from the foregoing, the inventor has demonstrated soluble protein can be extracted with a carboxylic acid which is inexpensive and which do not require specialized equipment for processing, storage and/or disposal. An exemplary carboxylic acid used by the inventor is acetic acid.

The inventor has shown that this method is effective across a wide range of concentrations of a carboxylic acid. The inventor has also shown that the amount of protein extracted and/or the purity of the extracted protein can be improved by increasing the ratio of the volume of acid to the weight of microorganisms.

The methods produced by the inventor do not necessarily require specialized equipment, e.g., homogenizers or sonicators, or harsh organic solvents or enzymes, such as lysozyme, to extract soluble protein.

The findings by the inventor provides the basis for methods for extracting soluble proteins from microorganisms, as well as methods for purifying proteins for use in humans or non-human animals.

Based on the foregoing discussion, the present disclosure provides a method for extracting a soluble protein from a population of microorganisms, the method comprising contacting the population of microorganisms expressing the soluble protein with a solution comprising an amount of a carboxylic acid effective to extract the soluble protein from the population of microorganisms.

In one example, the carboxylic acid is acetic acid.

In one example, the population of microorganisms is contacted with the solution for a time sufficient to extract the soluble proteins. For example, the population of microorganisms is contacted for less than about 48 hours or 24 hours or 20 hours, e.g., less than about 12 hours, such as less than about 8 hours or 6 hours. For example, the population of microorganisms is contacted for less than about 2 hours or 1 hour, e.g., for between about 20 minutes and 1 hour.

The present disclosure provides a method for extracting a soluble protein from a population of microorganisms, the method comprising contacting the population of microorganism expressing the soluble protein with an amount of a solution comprising from about 1% (v/v) to about 100% (v/v) carboxylic acid effective to extract the soluble protein from the population of microorganisms. For example, the solution comprises between about 1% and about 90% or 80% or 70% or 60% or 50% carboxylic acid. For example, the solution comprises between about 10% and about 90% or 80% or 70% or 60% or 50% carboxylic acid. For example, the solution comprises between about 20% and about 90% or 80% or 70% or 60% or 50% carboxylic acid. In some examples, the method comprises contacting the population with such a solution for a specified period of time (e.g., 1 hour or less) as described herein.

The present disclosure provides a method for extracting a soluble protein from a population of microorganisms, the method comprising contacting the population of microorganism expressing the soluble protein with an amount of a solution comprising from about 1% (v/v) to less than 50% (v/v) carboxylic acid effective to extract the soluble protein from the population of microorganisms.

In one example, the solution comprises from about 1% (v/v) to about 40% (v/v) carboxylic acid.

In one example, the solution comprises from about 1% (v/v) to about 37.5% (v/v) carboxylic acid.

In one example, the solution comprises from about 1% (v/v) to about 30% (v/v) carboxylic acid.

In one example, the solution comprises from about 1% (v/v) to about 25% (v/v) carboxylic acid.

For example, the solution comprises from about 10% (v/v) to about 40% (v/v) carboxylic acid.

For example, the solution comprises from about 10% (v/v) to about 37.5% (v/v) carboxylic acid.

For example, the solution comprises from about 10% (v/v) to about 25% (v/v) carboxylic acid.

For example, the solution comprises from about 20% (v/v) to about 40% (v/v) carboxylic acid.

For example, the solution comprises from about 20% (v/v) to about 37.5% (v/v) carboxylic acid.

For example, the solution comprises from about 25% (v/v) to about 37.5% (v/v) carboxylic acid.

Additional amounts of acetic acid are described herein and are to be taken to apply mutatis mutandis to the present example of the disclosure.

Using a solution comprising 40% or 37.5% or 25% or less carboxylic acid facilitates downstream purification with fewer steps and/or reduced cost. This is because several filters used for processing extracted protein, e.g., by tangential flow filtration, can be damaged by levels of carboxylic acid, e.g., acetic acid, greater than 40% or 37.5% or 25%. Exemplary filters damaged by these conditions comprise or consist of reconstituted cellulose or polyethersulfone.

The present disclosure also provides a method for extracting a soluble protein from a population of microorganisms, the method comprising contacting the population of microorganism expressing the soluble protein with an amount of a solution comprising a carboxylic acid effective to extract the soluble protein from the population of microorganisms for less than about 20 hours or 15 hours or 10 hours or 5 hours or 4 hours or 3 hours or 2 hours or 1 hour. In one example, the population of microorganism is contacted with the solution for less than about 1 hour, e.g., between about 10 minutes and about 1 hour. In one example, the population of microorganism is contacted with the solution for between about 20 minutes and about 1 hour.

Additional times of extraction are described herein and are to be taken to apply mutatis mutandis to the present example of the disclosure.

In one example, the solution comprises between about 10% and about 100% of carboxylic acid, e.g., between about 10% and 90% or 80% or 75% or 70% or an amount described herein.

In one example of a method of the present disclosure the carboxylic acid is added at a dissolution ratio from about 1:1 (milliliters of acid:grams of microorganism (wet)) to about 20:1 (milliliters of acid:grams of microorganism (wet)). For example, the carboxylic acid is added at a dissolution ratio from about 5:1 (milliliters of acid:grams of microorganism (wet)) to about 20:1 (milliliters of acid:grams of microorganism (wet)), such as to about 10:1 (milliliters of acid:grams of microorganism (wet)). In one exemplary form of the disclosure, the carboxylic acid is added at a dissolution ratio of about 9:1 (milliliters of acid:grams of microorganism (wet)).

In one example, the carboxylic acid is added to microorganisms that are not suspended in a solution, such as cell culture medium. For example, the microorganisms have been sedimented by centrifugation.

In one example, carboxylic acid is added to a solution comprising microorganisms, e.g., a cell culture medium or other solution, such that it comprises from about 1% (v/v) to less than 50% (v/v) carboxylic acid

Carboxylic acids will be apparent to the skilled artisan. Exemplary carboxylic acids are listed in Table 1.

In one example, the carboxylic acid has a pKa of at least about 3. As exemplified herein, suitable carboxylic acids include acetic acid or formic acid. The inventor has shown that acetic acid is useful for extracting soluble protein with a higher recovery and/or level of purity than urea or sodium hydroxide or isopropanol.

In one example and as appropriate, a method described herein according to any example comprises contacting the population of microorganisms with the solution for about five hours or less, such as four hours or less, for example 3 hours or less or two hours or less. In one example, the method comprises contacting the population of microorganisms with the solution for about 1 hour or less. The skilled artisan will understand that this method requires actual contacting of the population with the acid. Thus, the term “or less” means at least 1 minute, e.g., at least 5 or 10 or 15 minutes.

In one example, the method additionally comprises contacting the population of microorganisms with a detergent, such as Tween (polyoxyethylene-sorbitan monooleate, e.g., Tween 20 or Tween 80) or a detergent from the TritonX class of detergents (polyethylene glycol p-(1,1,3,3-tetramethylbutyl)-phenyl ether). In one example, the detergent is added with the carboxylic acid. In one example, the detergent is added after the carboxylic acid. In one example, the detergent is added before the carboxylic acid.

In one example, the soluble protein is recombinantly expressed by the population of microorganisms.

Exemplary microorganisms include bacteria, yeast or fungi. Exemplary microorganisms include bacteria, such as Gram negative bacteria, such as Escherichia coli.

In one example, the soluble protein has a pI that permits the protein to remain soluble when the protein is extracted from the population of microorganisms. In one example, the protein has a neutral pI, e.g., from about 6 to about 8, e.g., from about 6.5 to about 7.5.

In another example, the protein has an alkaline pI. For example, the soluble protein has a pI 7.5 or greater. In another example, the soluble protein has a pI of about 10.4.

In another example, the protein has an acidic pI. For example, the protein has a pI of about 6.5 or less. In another example, the protein has a pI of 6 or less or 5.5 or less. For example, the protein has a pI of 5 or less. For example, the protein has a pI of about 4.7 or less, e.g., about 4.65 or 4.6.

In one example, protein is selected from the group consisting of a scleroprotein, a chaperonin, a heat shock protein, a peptide (e.g., a natriuretic peptide) and a protein comprising a variable domain of an antibody.

In one example, the protein is a fusion protein. For example, the protein comprises multiple copies (e.g., two or three copies) of a peptide or polypeptide. In another example, the protein comprises two polypeptides fused to one another, e.g., a peptide or polypeptide fused to a tag to facilitate purification (e.g., a hexa-histidine tag) or a Fc region of an antibody (e.g., an IgG1 antibody).

In one example, the method additionally comprises removing a solution comprising the soluble protein from insoluble components of the population of microorganisms. For example, the method additionally comprises centrifuging and/or filtering the solution and recovering the soluble fraction, e.g., supernatant from centrifugation.

The present disclosure also provides a method for purifying a soluble protein from a population of microorganisms, the method comprising performing the method for extracting a protein as described herein and purifying the soluble protein extracted by the method.

In one example, a method described herein additionally comprises modifying the purified or extracted protein. For example, the protein can be modified by conjugating it to another protein or cleaving it (e.g., to remove a tag and/or to cleave multiple copies of a peptide within a fusion protein).

The present disclosure additionally provides a method for producing a modified protein, the method comprising obtaining the protein purified by a method as described herein or a protein extracted by a method described herein and modifying the protein. In one example, modifying the protein comprises conjugating another compound to the protein. In another example, modifying the protein comprises contacting it with a protease to thereby cleave the protein.

In one example, a method as described herein additionally comprises formulating the therapeutic protein into a pharmaceutical composition or a cosmetic composition, or a veterinary composition.

In one example, a method as described herein additionally comprises immobilizing the protein onto or into a solid support or semi-solid support. Exemplary supports are patches and/or implants and/or medical devices.

The present disclosure also provides a method for producing a pharmaceutical composition or a cosmetic composition, or a veterinary composition, the method comprising obtaining the protein extracted, purified or produced by the method described herein and formulating the protein into a pharmaceutical composition or a cosmetic composition, or a veterinary composition.

The present disclosure also provides a method for producing a solid or semi-solid support, the method comprising obtaining the protein extracted, purified or produced by a method described herein according to any example and immobilizing the protein onto or into a solid support or semi-solid support.

In one example, the composition, solid support or semi-solid support is for administration or application to or on a human or a non-human animal.

In one example, the composition, solid support or semi-solid support is for use as a medicament or as a cosmetic.

In one example, the composition, solid support or semi-solid support is for pharmaceutical use, cosmetic use, cosmaceutical use, veterinary use or for implantation.

In one example, the composition, solid support or semi-solid support is for administration orally, by implantation, intra-dermally, intramuscularly, subcutaneously, intravenously, intra-arterially, intra-muscularly, as an aerosol, or intra-ocularly.

The present disclosure additionally provides a protein or composition or solid support or semi-solid support produced by performing a method described herein.

In one example, the protein (including modified protein), composition, solid support or semi-solid support is for pharmaceutical use, cosmetic use, cosmaceutical use, veterinary use or for implantation. In one example, the composition is a pharmaceutical composition. In one example, the composition is a cosmetic composition. Methods described herein are also useful for producing scaffolds for growing or immobilizing cells, e.g., stem or progenitor cells or skin cells, or for producing enzymes for industrial application, e.g., in the food or textile industries.

The present disclosure also provides a device comprising the protein, composition, solid support or semi-solid support described herein. In one example, the device is a syringe, a patch or an implant.

### BRIEF DESCRIPTION OF THE DRAWINGS

FIG. 1 is a copy of a photographic representation showing results of polyacrylamide gel electrophoresis analysis of various treatments to extract a recombinant protein from E. coli. Lane 1, size markers; Lane 2, 15 mM NaOH extraction; Lane 3, 0.57 (v/v) acetic acid extraction (room temperature); Lane 4, 0.57% (v/v) acetic acid extraction (4° C.); Lane 5, 10% (w/v) formic acid extraction; Lane 6, urea extraction; Lane 7 n-propanol extraction.

FIG. 2 is a copy of a photographic representation showing results of polyacrylamide gel electrophoresis analysis of various treatments to extract a recombinant protein from E. coli. Lane 1, size markers; Lane 2, 10% (v/v) acetic acid extraction; Lane 3, 5% (v/v) acetic acid extraction; Lane 4, (2% (v/v) acetic acid extraction; Lane 5, urea extraction.

FIG. 3 is a graphical representation showing the relationship between the concentration of acetic acid (v/v), the dissolution ratio and the amount (g) of recombinant protein recovered per liter of ferment.

FIG. 4 is a graphical representation showing the relationship between the concentration of acetic acid (v/v), the dissolution ratio and the purity of recombinant protein extracted.

FIG. 5 is a copy of a photographic representation showing results of SDS-PAGE analysis of extracts of E. coli expressing a recombinant protein. The extracts were produced using different concentrations of acetic acid (v/v) as indicated at the top of the Figure.

FIG. 6 is a graphical representation showing the yield and purity of recombinant protein in extracts of E. coli as determined using RP-HPLC. The extracts were produced using different concentrations of acetic acid (v/v) as indicated.

FIG. 7 includes two graphical representations showing yield (left-hand side) and purity (right-hand side) of recombinant protein in extracts of E. coli as determined using RP-HPLC. The extracts were produced using different concentrations of acetic acid (v/v) and over different periods of time as indicated.

FIG. 8 is a copy of a photographic representation showing results of SDS-PAGE analysis of extracts of E. coli expressing a recombinant protein. The extracts were produced using different concentrations of acetic acid (v/v) as indicated at the top of the Figure over a 20 minute period.

FIG. 9 is a copy of a photographic representation showing results of SDS-PAGE analysis of extracts of E. coli expressing a recombinant fusion protein containing multiple copies of a peptide. Lane 1, size markers; Lane 2 cell lysate; and Lane 3, 25% (v/v) acetic acid extraction.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the present disclosure is susceptible to variations and modifications other than those specifically described. It is to be understood that the disclosure encompasses all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only.

Functionally-equivalent products, compositions and methods are clearly within the scope of the disclosure.

Any example herein shall be taken to apply mutatis mutandis to any other embodiment unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein and cell culture techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### SELECTED DEFINITIONS

The term “extract” means removing a soluble protein from the microorganism in which it is expressed. In some examples, extracting a soluble protein removes the protein from at least about 10%, about 20%, about 30%, about 40%, about 50%, about 60%, about 70%, of the other materials or components that were present prior to the extracting, e.g., in the microorganism.

As used herein, the term “soluble protein” will be understood to mean that when expressed within a microorganism (e.g., within the cytosol or periplasm of the microorganism, the protein is in soluble form). For example, the protein does not form insoluble aggregates, e.g., inclusion bodies, in the cytosol or periplasm of a cell.

The term “microorganism” will be apparent to the skilled person as referring to any microscopic unicellular or multicellular organism. Exemplary microorganisms are unicellular. Examples of microorganisms useful in the method of the present disclosure include yeast, fungi and bacteria.

The term “population of microorganisms” includes microorganisms produced during culture or fermentation. In one example, the microorganisms in the culture arise from a clone, i.e., are clonal. For example, the cells are substantially genetically identical, other than mutations that arise during the culture process.

As used herein, the term “carboxylic acid” encompasses any organic acid comprising at least one carboxyl group, including the carboxylic acids listed in Table 1. In one example, the carboxylic acid has a pKa greater than 2 or 2.5 or 3 or 3.5 or 3.6 or 3.7 or 3.8 or 3.9 or 4 or 4.1 or 4.2 or 4.3 or 4.4 or 4.5. For example, the carboxylic acid is acetic acid or formic acid or citric acid or lactic acid or uric acid or benzoic acid or chloroacetic acid or propanoic acid. In one example, the carboxylic acid is formic acid or acetic acid or benzoic acid or propanoic acid. In one example, the carboxylic acid is acetic acid or benzoic acid or propanoic acid. In one example, the carboxylic acid is acetic acid.

The skilled person will understand that the term “% (v/v)” (syn. “volume percent” or “volume for volume percent”) will be understood to be calculated by the formula ((total volume of solute (carboxylic acid)/total volume of the solution)×100%).

The term “wet weight” simply means that the weight of the population of microorganisms is measured without first drying the microorganisms. For example, the weight of the microorganisms is determined after harvesting from cell culture (e.g., by centrifugation and/or filtration), however without drying the cells.

The meaning of the term “dissolution ratio” will be apparent to the skilled person from the description herein. In particular, this term refers to the ratio of the volume of solution comprising carboxylic acid to the wet weight of the population of microorganisms.

### Carboxylic Acids

As discussed herein, the present disclosure contemplates the use of any carboxylic acid to extract a soluble protein from a cell.

In one example, the carboxylic acid has a sufficiently low pKa to permit extraction when used at a % (v/v) discussed herein.

In one example, the carboxylic acid has a sufficiently high pKa that it does not damage the equipment used to extract the protein, e.g., the reaction vessel, when used at a % (v/v) of from about 1% to less than 50%, such as from about 1% to about 37.5%.

Exemplary carboxylic acids and their pKas are set forth in Table 1.

TABLE 1
Exemplary carboxylic acids and their pKas.

| Molecular | | |
|---|---|---|
| formula | Name | pKₐ |
| CH2O2 | Formic acid | 3.75 |
| C2HCl3O2 | Trichloroacetic acid | 0.70 |
| C2H2Cl2O2 | Dichloroacetic acid | 1.48 |
| C2H2O4 | Oxalic acid | 1.23 |
| C2H3BrO2 | Bromoacetic acid | 2.69 |
| C2H3ClO2 | Chloroacetic acid | 2.85 |
| C2H3IO2 | Iodoacetic acid | 3.12 |
| C2H4OS | Thioacetic acid | 3.33 |
| C2H4O2 | Acetic acid | 4.76 |
| C2H4O3 | Glycolic acid | 3.83 |
| C3H4O2 | Acrylic acid | 4.25 |
| C3H6O2 | Propanoic acid | 4.86 |
| C3H6O3 | Lactic acid | 3.08 |
| C3H6O4 | Glyceric acid | 3.52 |
| C4H4O4 | Maleic acid | 1.83 |
| C4H4O5 | Oxaloacetic acid | 2.22 |
| C4H6O3 | Acetoacetic acid | 3.58 |
| C4H6O4 | Succinic acid | 4.16 |
| C4H6O5 | Malic acid | 3.40 |
| C4H7NO4 | Aspartic acid | 1.99 |
| C4H8O2 | Butanoic acid | 4.83 |
| C4H8O2 | 2-Methylpropanoic acid | 4.88 |
| C4H8O3 | 3-Hydroxybutanoic acid | 4.70 |
| C4H8O3 | 4-Hydroxybutanoic acid | 4.72 |
| C4H9NO2 | 2-Aminobutanoic acid | 2.29 |
| C4H9NO2 | 4-Aminobutanoic acid | 4.031 |
| C5H4N4O3 | Uric acid | 3.89 |
| C5H8O4 | Glutaric acid | 4.31 |
| C5H9NO4 | L-Glutamic acid | 2.13 |
| C5H10O2 | Pentanoic acid | 4.84 |
| C5H10O2 | Trimethylacetic acid | 5.03 |
| C6H5NO2 | Picolinic acid | 1.07 |
| C6H8O7 | Citric acid | 3.14 |
| C6H8O7 | Isocitric acid | 3.29 |
| C6H10O4 | Adipic acid | 4.43 |
| C6H11NO3 | Adiparnic acid | 4.63 |
| C6H12O2 | Hexanoic acid | 4.85 |
| C7H5BrO2 | 2-Bromobenzoic acid | 2.84 |
| C7H5BrO2 | 3-Bromobenzoic acid | 3.86 |
| C7H5CIO2 | 2-Chlorobenzoic acid | 2.92 |
| C7H5CIO2 | 3-Chlorobenzoic acid | 3.82 |
| C7H5CIO2 | 4-Chlorobenzoic acid | 3.98 |
| C7H5IO2 | 2-Iodobenzoic acid | 2.85 |
| C7H5IO2 | 3-Iodobenzoic acid | 3.80 |
| C7H5NO4 | Quinolinic acid | 2.43 |
| C7H6O2 | Benzoic acid | 4.19 |
| C7H14O2 | Heptanoic acid | 4.89 |
| C8H16O2 | Octanoic acid | 4.89 |

In one example, the carboxylic acid has a pKa greater than 2. For example, the carboxylic acid is pentanoic acid, trimethylacetic acid, citric acid, isocitric acid, adipic acid, adiparnic acid, hexanoic acid, 2-bromobenzoic acid, 3-bromobenzoic acid, 2-chlorobenzoic acid, 3-chlorobenzoic acid, 4-chlorobenzoic acid, 2-iodobenzoic acid, 3-iodobenzoic acid, quinolinic acid, benzoic acid, heptanoic acid, octanoic acid formic acid, bromoacetic acid, chloroacetic acid, iodoacetic acid, thioacetic acid, acetic acid, glycolic acid, acrylic acid, propanoic acid, lactic acid, glyceric acid, oxaloacetic acid, acetoacetic acid, succinic acid, malic acid, butanoic acid, 2-methylpropanoic acid, 3-hydroxybutanoic acid, 4-hydroxybutanoic acid, 2-aminobutanoic acid, 4-aminobutanoic acid, uric acid, glutaric acid or 1-glutamic acid.

In one example, the carboxylic acid has a pKa greater than 3. For example, the carboxylic acid is formic acid, iodoacetic acid, thioacetic acid, acetic acid, glycolic acid, acrylic acid, propanoic acid, lactic acid, glyceric acid, acetoacetic acid, succinic acid, malic acid, butanoic acid, 2-methylpropanoic acid, 3-hydroxybutanoic acid, 4-hydroxybutanoic acid, 4-aminobutanoic acid, uric acid, glutaric acid, pentanoic acid, trimethylacetic acid, citric acid, isocitric acid, adipic acid, adiparnic acid, hexanoic acid, 3-chlorobenzoic acid, 4-chlorobenzoic acid, 3-iodobenzoic acid, benzoic acid, heptanoic acid, or octanoic acid.

In one example, the carboxylic acid has a pKa greater than 4. For example, the carboxylic acid is acetic acid, acrylic acid, propanoic acid, succinic acid, butanoic acid, 2-methylpropanoic acid, 3-hydroxybutanoic acid, 4-hydroxybutanoic acid, 2-aminobutanoic acid, 4-aminobutanoic acid, glutaric acid, pentanoic acid, adipic acid, hexanoic acid, heptanoic acid, octanoic acid or benzoic acid.

In one example, the carboxylic acid has a pKa greater than 4.5 For example, the carboxylic acid is acetic acid, propanoic acid, butanoic acid, 2-Methylpropanoic acid, 3-Hydroxybutanoic acid, 4-Hydroxybutanoic acid, pentanoic acid, trimethylacetic acid, adiparnic acid, hexanoic acid or oxanoic acid.

In one example, the carboxylic acid is acetic acid or formic acid.

In one exemplary form of the disclosure, the carboxylic acid is acetic acid. The inventor has shown that this acid provides superior extraction properties (e.g., yield and/or purity) even compared to another carboxylic acid (formic acid). This carboxylic acid is also relatively inexpensive (e.g., compared to some organic solvents) and generally does not require specialized equipment for processing, storage or disposal.

In examples of the disclosure in which the population of microorganisms is contacted with the composition for a defined period of time, the carboxylic acid is used at a % (v/v) of from about 1% to less than 100%. For example, the carboxylic acid is used at a % (v/v) of from about 1% to about 95%. For example, the carboxylic acid is used at a % (v/v) of from about 1% to about 90%. For example, the carboxylic acid is used at a % (v/v) of from about 1% to about 85%. For example, the carboxylic acid is used at a % (v/v) of from about 1% to about 80%. For example, the carboxylic acid is used at a % (v/v) of from about 1% to about 75%. For example, the carboxylic acid is used at a % (v/v) of from about 1% to about 70%. For example, the carboxylic acid is used at a % (v/v) of from about 1% to about 65%. For example, the carboxylic acid is used at a % (v/v) of from about 1% to about 60%. For example, the carboxylic acid is used at a % (v/v) of from about 1% to about 55%.

In one example of a method of the disclosure, the carboxylic acid is used at a % (v/v) of from about 1% to less than 50%. For example, the carboxylic acid is used at a % (v/v) of from about 1% to about 45%. For example, the carboxylic acid is used at a % (v/v) of from about 2% to about 50%. For example, the carboxylic acid is used at a % (v/v) of from about 2% to about 40%. For example, the carboxylic acid is used at a % (v/v) of from about 2% to about 37.5%. For example, the carboxylic acid is used at a % (v/v) of from about 3% to about 37.5%. For example, the carboxylic acid is used at a % (v/v) of from about 3% to about 30%. For example, the carboxylic acid is used at a % (v/v) of from about 5% to about 30%. For example, the carboxylic acid is used at a % (v/v) of from about 6% to about 30%. For example, the carboxylic acid is used at a % (v/v) of from about 7% to about 30%. For example, the carboxylic acid is used at a % (v/v) of from about 8% to about 30%. For example, the carboxylic acid is used at a % (v/v) of from about 9% to about 30%. For example, the carboxylic acid is used at a % (v/v) of from about 10% to about 37.5%. For example, the carboxylic acid is used at a % (v/v) of from about 10% to about 25%. For example, the carboxylic acid is used at a % (v/v) of 1% or 2% or 5% or 10% or 15% or 17.5% or 25% or 37.5%.\ For example, the carboxylic acid is used at a % (v/v) of 25%.

In one example, the carboxylic acid is diluted in a solution that does not have a significant or detectable buffering capacity.

In one example, the carboxylic acid is diluted in water.

In one example, the solution comprising the carboxylic acid has a pH of between 2 and 6, for example, between 2 and 5, such as between 2 and 4. For example, the solution comprising the carboxylic acid has a pH of between 2 and 3. In one example, solution comprising the carboxylic acid has a pH of between 2 and 2.5.

### Extraction

In one example, the carboxylic acid is added at a dissolution ratio from about 1:1 (milliliters of acid:grams of microorganism (wet)) to about 20:1 (milliliters of acid:grams of microorganism (wet)). For example, the carboxylic acid is added at a dissolution ratio from about 2:1 (milliliters of acid:grams of microorganism (wet)) to about 15:1 (milliliters of acid:grams of microorganism (wet)). For example, the carboxylic acid is added at a dissolution ratio from about 3:1 (milliliters of acid:grams of microorganism (wet)) to about 10:1 (milliliters of acid:grams of microorganism (wet)). The carboxylic acid is added at a dissolution ratio from about 5:1 (milliliters of acid:grams of microorganism (wet)) to about 10:1 (milliliters of acid:grams of microorganism (wet)). For example, the carboxylic acid is added at a dissolution ratio of about 5:1 (milliliters of acid:grams of microorganism (wet)) or about 6:1 (milliliters of acid:grams of microorganism (wet)) or about 7:1 (milliliters of acid:grams of microorganism (wet)) or about 8:1 (milliliters of acid:grams of microorganism (wet)) or about 9:1 (milliliters of acid:grams of microorganism (wet)).

In one exemplary form of the disclosure, the carboxylic acid is added at a dissolution ratio of about 9:1 (milliliters of acid:grams of microorganism (wet)).

It will be apparent to the skilled person that in some examples it is desirable to harvest the microorganisms prior to weighing the microorganisms. Methods for harvesting microorganisms will be apparent to the skilled artisan and include, for example, continuous flow centrifugation (e.g., using a disc-stack centrifuge, such as those available from Alfa Laval or GEA Westfalia Separator Group GmbH), microfiltration or tangential flow filtration.

Following harvesting the weight of the microorganisms is readily determined. Alternatively, the weight of a sample of the microorganisms is determined and this weight is used to calculate or estimate the weight of the entire population of microorganisms.

In an alternative example, a sample of a culture of microorganisms is harvested and weighed and this weight is used to calculate or estimate the weight of microorganisms in a culture. The desired amount of carboxylic acid containing solution can then be directly added to the culture.

In a further example, the weight of a culture of microorganisms is estimated based on the known weights of each of the components added to the culture (e.g., media and feeds) and these weights are used to calculate or estimate the weight of microorganisms in a culture. The desired amount of carboxylic acid containing solution can then be directly added to the culture.

In one example, the population of microorganisms is contacted with the solution for a time and under conditions sufficient to extract the soluble protein.

As discussed herein above, the inventor has determined that a method disclosed herein according to any example facilitates rapid extraction of a soluble protein from a population of microorganisms. For example, the inventor found that a similar amount of protein was extracted from a population of microorganisms when it was contacted with a solution comprising a carboxylic acid for about 20 minutes or about 1 hour or about 20 hours. In one example, the method comprises contacting a population of microorganisms with a solution comprising a carboxylic acid for less than five hours or four hours or three hours or two hours. For example, the method comprises contacting a population of microorganisms with a solution comprising a carboxylic acid for one hour or less, such as 45 minutes or 30 minutes or 20 minutes. The present disclosure also contemplates longer periods of extraction. For example, the population of microorganisms is contacted with the carboxylic acid for from about 1 hour to about 20 hours, such as from 1 to 5 hours, e.g., from 1 to 3 hours. In one example, the population of microorganisms is contacted with the carboxylic acid for about 1 hour.

The population of microorganisms can be contacted with the carboxylic acid at a temperature of between 2° C. and 40° C., such as between 4° C. to about 30° C., for example, at room temperature. The skilled artisan will understand that room temperature will depend on the environment in which the extraction is performed. For example, in a manufacturing facility, the temperature can be between 16° C. to 26° C., such as between 18° C. to 25° C., e.g., 19° C. or 20° C. or 21° C. or 22° C. or 23° C. or 24° C.

The solution comprising the carboxylic acid and population of microorganisms can be agitated using standard techniques for mixing.

Following contacting with the carboxylic acid, solution containing soluble protein can be separated from contaminants. For example, the solution containing the carboxylic acid, population of microorganisms and soluble protein can be subjected to centrifugation and/or microfiltration. The soluble fraction, which contains the soluble protein, is then recovered.

In one example, the extraction method described herein does not comprise contacting the population of microorganisms with a mineral acid.

In one example, the extraction method described herein does not comprise contacting the population of microorganisms with an organic solvent, other than the carboxylic acid or water. In one example, the method does not comprise contacting the population of microorganisms with an alcohol, e.g., butanol or propanol.

In one example, the extraction method described herein does not comprise sonication and/or homogenization.

In one example, the extraction method described herein does not comprise addition of a solubilization enhancer such as polyethyleneimine, magnesium sulfate (MgSO₄), magnesium chloride (MgCl₂), calcium sulfate (CaSO₄), or calcium chloride (CaCl₂).

### Micororganisms

Suitable microorganisms for expressing a soluble protein for extraction according to the present disclosure will be apparent to the skilled artisan. For example, the microorganism is a yeast, a fungus or a bacterium.

Exemplary yeast include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, and Candida albicans.

Exemplary filamentous fungi include Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

Suitable bacteria for use in the present disclosure include archaebacteria and eubacteria, especially eubacteria. For example, Gram-negative bacteria, such as Enterobacteriaceae are useful in the methods of the present disclosure. Examples of useful bacteria include Escherichia, Enterobacter, Azotobacter, Envinia, Bacillus, Pseudomonas, Klebsiella, Proteus, Salmonella, Serratia, Shigella, Rhizobia, Vitreoscilla, and Paracoccus.

In one example, the microorganism is Escherichia coli. Suitable E. coli hosts include E. coli W3110 (ATCC 27,325), E. coli 294 (ATCC 31,446 or 33,625), E. coli B, and E. coli X1776 (ATCC 31,537). These examples are illustrative rather than limiting. Mutant cells of any of the above-mentioned microorganisms may also be employed. Suitable microorganisms can be selected by taking into consideration replicability of an expression vector in the microorganism, when taking advantage of recombinant expression. For example, E. coli, Serratia, or Salmonella species can be suitably used as the host when known plasmids such as pBR322, pBR325, pACYC 177, or pKN410 are used.

When extracting an endogenous protein, it is important to select a microorganism expressing the protein.

### Recombinant Expression

In one example, a soluble protein is expressed recombinantly, i.e., using recombinant processes/means. For example, a nucleic acid encoding the soluble protein is operably linked to a promoter in an expression construct. The expression construct can be introduced into the microorganism where it can incorporate into the genome of the microorganism or a plasmid therein or can remain episomal. In some examples, the expression construct is an expression vector.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Preferred promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

The term “expression construct” is to be taken in its broadest context and includes a nucleic acid comprising a promoter operably linked to a nucleic acid encoding a soluble protein and any other elements required for expression of the soluble protein.

The term “expression vector” refers to a nucleic acid comprising at least a promoter operably linked to a nucleic acid encoding a soluble protein and any other elements required for expression of the soluble protein as well as sequences that permit the vector to replicate within a microorganism and, optionally a sequence encoding a selectable marker. Within the context of the present invention, it is to be understood that an expression vector may be a plasmid, bacteriophage, phagemid, cosmid, virus sub-genomic or genomic fragment, or other nucleic acid capable of maintaining and or replicating heterologous DNA in an expressible format. Many expression vectors are commercially available for expression in a variety of cells. Selection of appropriate vectors is within the knowledge of those having skill in the art.

Typical promoters suitable for expression in viruses of bacterial cells and bacterial cells such as for example a bacterial cell selected from the group comprising E. coli, Staphylococcus sp, Corynebacterium sp., Salmonella sp., Bacillus sp., and Pseudomonas sp., include, but are not limited to, the lacz promoter, the Ipp promoter, temperature-sensitive λ_{L} or λ_{R} promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, such as for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio-TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer nc., CT, USA); the pQE series of expression vectors (QIAGEN, CA, USA), or the pL series of expression vectors (Invitrogen), amongst others.

Typical promoters suitable for expression in yeast cells such as for example a yeast cell selected from the group comprising Pichia pastoris, S. cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Expression vectors for expression in yeast cells include, but are not limited to, the pACT vector (Clontech), the pDBleu-X vector, the pPIC vector suite (Invitrogen), the pGAPZ vector suite (Invitrogen), the pHYB vector (Invitrogen), the pYD1 vector (Invitrogen), and the pNMT1, pNMT41, pNMT81 TOPO vectors (Invitrogen), the pPC86-Y vector (Invitrogen), the pRH series of vectors (Invitrogen), pYESTrp series of vectors (Invitrogen). A number of other expression constructs and methods for their use are known in the art and are described for example, in Giga-Hama and Kumagai (In: Foreign Gene Expression in Fission Yeast: Schizosaccharomyces pombe, Springer Verlag, ISBN 3540632700, 1997) and Guthrie and Fink (In: Guide to Yeast Genetics and Molecular and Cell Biology Academic Press, ISBN 0121822540, 2002).

Expression constructs or expression vectors are introduced into microoganisms using standard methods know in the art. For example, treatment employing multivalent cations (e.g., calcium), as described in Sambrook et al., Molecular Cloning: A Laboratory Manual (New York: Cold Spring Harbor Laboratory Press, 1989), is generally used for bacterial cells that contain substantial cell-wall barriers. Another method for transformation employs polyethylene glycol/DMSO, as described in Chung and Miller, Nucleic Acids Res., 16: 3580 (1988). Other methods include electroporation, transfection using cationic lipids, viral delivery and mating strategies for fungi.

Following production of a recombinant microorganism, the microorganism is cultured under conditions sufficient to produce a population of microorganisms expressing the soluble protein. Thus, the method of the present disclosure also encompasses producing a recombinant cell and/or expressing the soluble protein.

Microorganisms used to produce the soluble protein are cultured in suitable media in which the promoters can be induced as described generally, e. g., in Sambrook et al., supra and/or Ausubel et al., supra and/or as are commercially available. Any other necessary supplements besides carbon, nitrogen, and inorganic phosphate sources may also be included at appropriate concentrations, introduced alone or as a mixture with another supplement or medium such as a complex nitrogen source. The pH of the medium may be any pH from about 5-9, depending mainly on the host organism. For accumulation of the soluble protein, the microorganism is cultured under conditions sufficient for accumulation of the soluble protein. Such conditions include, e. g., temperature, nutrient, and cell-density conditions that permit protein expression and accumulation by the microorganism. Moreover, such conditions are those under which the microorganism can perform basic cellular functions of transcription, translation, and passage of proteins from one cellular compartment to another, as are known to those skilled in the art.

In the case of inducible expression, e.g., making use of an inducible promoter, typically the cells are cultured until a certain optical density is achieved, at which point induction is initiated (e. g., by addition of an inducer, by depletion of a repressor, suppressor, or medium component, etc.), to induce expression of the gene encoding the soluble protein.

### Proteins

The present disclosure encompasses extraction of any protein that remains soluble in the microorganism, e.g., does not form an inclusion body in bacteria.

In one example, the soluble protein has a pI that permits the protein to remain soluble when the protein is extracted from the population of microorganisms.

In one example, the soluble protein has a pI of about 7.5 or more or 8 or more or 8.5 or more or 9 or more or 9.5 or more or 10 or more or 10.5 or more or 11 or more or 11.5 or more or 12 or more or 12.5 or more or 13 or more.

In one example, the soluble protein has a pI of about 6.5 or less or 6 or less or 5.5 or less or 5 or less or 4.5 or less or 4 or less or 3.5 or less or 3 or less or 2.5 or less or 2 or less. For example, the protein has a pI between about 3 and 6, such as between about 4 and 5, for example about 4.6.

In one example, the soluble protein has a pI of about 7, e.g., between about 6 and 7.

Methods for determining or estimating the pI of a protein will be apparent the skilled artisan. For example, the theoretical pI of a protein can be predicted using the method as described in Bjellqvist et al., Electrophoresis, 14: 1023-1031, 1993. The algorithm described by Bjellqvist et at is implemented in the Compute pI tool available from the ExPasy Proteomics Server available from the Swiss Institute of Bioinformatics.

Alternatively, the pI of the protein is determined using isoelectric focusing (IEF). IEF involves adding an ampholyte solution into immobilized pH gradient (IPG) gels. IPGs are the acrylamide gel matrix co-polymerized with the pH gradient. An electric current is passed through the gel, creating a “positive” anode and “negative” cathode end. Negatively charged proteins migrate through the pH gradient in the medium toward the “positive” end while positively charged proteins move toward the “negative” end. As a protein moves towards the pole opposite of its charge it moves through the changing pH gradient until it reaches a point in which the pH of that proteins isoelectric point is reached. At this point the protein no longer has a net electric charge (due to the protonation or deprotonation of the associated functional groups) and as such will not proceed any further within the gel.

In one example, the protein is used for therapeutic purposes, e.g., in humans. Exemplary proteins include recombinant human interleukin-11 (Opreleukin; pI 11.85), interferon (alfacon 1; pI9), interferon beta (pI 8.9), interferon gamma, tissue plasminogen activator (tPA), urokinase, octreotide (pI 8.29) or keratinocyte growth factor (pI 10.42).

In one example, the protein is an interferon, e.g., interferon beta.

In one example, the protein is an interleukin, e.g., a lymphokine, such as, IL-2.

In one example, the protein comprises a variable domain of an antibody. Exemplary variable domain containing proteins include, for example, domain antibodies (e.g., U.S. Pat. No. 6,248,516), a Fab fragment, a Fab′ fragment, a F(ab′) fragment, a scFv (e.g., as described in U.S. Pat. No. 5,260,203), a diabody, a triabody, a tetrabody or higher order complex (e.g., as described in WO98/044001 and/or WO94/007921). For example, the protein is a Fab fragment of an antibody. For example, the protein is abciximab, ranibizumab or certolizumab (which can be conjugated to polyethylene glycol to produce certolizumab pegol).

In one example, the protein is a scleroprotein. For example, the protein is a collagen (e.g., a type I collagen, a type II collagen, a type III collagen, a type IV collagen, a type V collagen, a type VI collagen, a type VII collagen, a type VIII collagen, a type IX collagen, a type X collagen, a type XI collagen or a type XII collagen), a tenascin (e.g., Tenascin C or Tenascin X), a laminin (e.g., laminin alpha, lamining beta or laminin gamma), fibrillin, ALCAM, vitronectin, decorin, matrix gla protein, elastin, tropoelastin or tectorin.

In one example the protein comprises or has a coiled-coil structure. For example, the protein is selected from the group consisting of fibrinogen, un-cross-linked keratin (epidermin), myosin, tropomyosin and a collagen.

In another example, the protein is a chaperonin or a heat shock protein. In one example, the protein is a type I chaperonin or a type II chaperonin Exemplary chaperonins are described, for example in Hill et al., Genome Res. 14:1669-1675, 2004. In one example, the protein is chaperonin 10, e.g., as described in U.S. Pat. No. 7,618,935.

In another example, the soluble protein is a natriuretic peptide, or an active fragment thereof.

In a further example, the soluble protein is an immunogenic fragment of a protein, e.g., as used in a vaccine.

In one example, the protein is a fusion protein.

For example, such a fusion protein can comprise multiple copies of a peptide or polypeptide, e.g., to facilitate large-scale production.

In another example, the fusion protein comprises a tag, e.g., to facilitate purification or detection, e.g., a hexa-His tag.

In a further example, the fusion protein comprises two peptides or polypeptides fused to one-another. For example, the fusion protein comprises a peptide or polypeptide fused to a Fc region of an antibody, e.g., an IgG antibody. For example, the fusion protein comprises one or more (e.g., two) thrombopoietin receptor binding domains fused to a Fc region of an antibody. For example, the fusion protein is romiplostim.

Reference herein to a protein includes mutants of the protein, e.g., comprising one or more conservative amino acid substitutions one or more deletions and/or one or more insertions. In one example, the protein contains fewer than 20 substitutions and/or deletions and/or insertions.

### Protein Modifications

The proteins extracted or recovered by the method of the present disclosure can be modified, e.g., by conjugation to another compound.

For example, the other compound is selected from the group consisting of a radioisotope, a detectable label, a therapeutic compound, a colloid, a toxin, a nucleic acid, a peptide, a protein, a compound that increases the half life of the protein in a subject and mixtures thereof.

Exemplary compounds are set forth in Table 2.

TABLE 2
Compounds useful in conjugation.

| Group | Detail |
|---|---|
| Radioisotopes (either | ¹²³I, ¹²⁵I, ¹³⁰I, ¹³³I, ¹³⁵I, ⁴⁷Sc, |
| directly or indirectly) | ⁷²As, ⁷²Sc, ⁹⁰Y, ⁸⁸Y, ⁹⁷Ru, ¹⁰⁰Pd, |
| | ^{101m}Rh, ^{101m}Rh, ¹¹⁹Sb, ¹²⁸Ba, ¹⁹⁷Hg, |
| | ²¹¹At, ²¹²Bi, ¹⁵³Sm, ¹⁶⁹Eu, |
| | ²¹²Pb, ¹⁰⁹Pd, ¹¹¹In, ⁶⁷Gu, ⁶⁸Gu, |
| | ⁶⁷Cu, ⁷⁵Br, ⁷⁶Br, ⁷⁷Br, ^{99m}Tc, |
| | ¹¹C, ¹³N, ¹⁵O, ¹⁸I, ¹⁸⁸Rc, ²⁰³Pb, |
| | ⁶⁴Cu, ¹⁰⁵Rh, ¹⁹⁸Au, ¹⁹⁹Ag or |
| | ¹⁷⁷Lu |
| Half life extenders | Polyethylene glycol |
| | Glycerol |
| | Glucose |
| | albumin |
| Fluorescent probes | Phycoerythrin (PE) |
| | Allophycocyanin (APC) |
| | Alexa Fluor 488 |
| | Cy5.5 |
| Biologics | Fluorescent proteins such as |
| | Renilla luciferase, GFP |
| | Immune modulators |
| | Toxins |
| | An Immunoglobulin |
| Chemotherapeutics | Taxol |
| | 5-FU |
| | Doxorubicin |
| | Idarubicin |

### Protein Purification

In some examples, the method of the present disclosure comprises additionally purifying the protein. Various methods are known in the art for purifying proteins.

The protein prepared from the microorganisms can be purified using, for example, ion exchange chromatography, hydroxyapatite chromatography, fluoroapatite chromatography, displacement chromatography, gel electrophoresis, dialysis, ultrafiltration or affinity chromatography. These techniques are known in the art and described, for example, in Scopes (In: Protein purification: principles and practice, Third Edition, Springer Verlag, 1994). Other techniques for protein purification such as ethanol precipitation, Reverse Phase HPLC, chromatography on silica, chromatography on heparin, chromatofocusing, SDS-PAGE, and ammonium sulfate precipitation are also available depending on the protein to be recovered.

The skilled artisan will also be aware that a soluble protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. Preferably, the tag is a hexa-his tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein.

### Formulation

A protein extracted and/or purified as described herein can be formulated into a composition. For example, the compositions can be for parenteral administration, topical administration, oral administration, intramusclular administration, intraocular administration, subcutaneous administration, local administration, aerosol administration, intradermal administration or transdermal administration for prophylactic or for therapeutic treatment or for cosmetic treatment.

Typically, a therapeutically effective amount of the protein will be formulated into a composition for administration to a subject. The phrase “a therapeutically effective amount” refers to an amount sufficient to promote, induce, and/or enhance treatment or other therapeutic effect in a subject. As will be apparent, the concentration of protein in such a composition can vary widely, and will be selected primarily based on fluid volumes, viscosities, body weight and the like in accordance with the particular mode of administration selected and the patient's needs. Depending on the type and severity of the disease, a therapeutically effective amount may be about 1 μg/kg to 150 mg/kg of protein, whether, for example, by one or more separate administrations, or by continuous infusion. A typical daily dosage might range from about 1 μg/kg to 100 mg/kg or more. An exemplary dosage of the protein to be administered to a patient is in the range of about 0.1 to about 10 mg/kg of patient weight.

The compositions for administration will commonly comprise a solution of the protein dissolved in a pharmaceutically acceptable carrier, preferably an aqueous carrier. A variety of aqueous carriers can be used, e.g., buffered saline and the like. Other exemplary carriers include water, saline, Ringer's solution, dextrose solution, and 5% human serum albumin. Nonaqueous vehicles such as mixed oils and ethyl oleate may also be used. Liposomes may also be used as carriers. The carriers may contain minor amounts of additives that enhance isotonicity and chemical stability, e.g., buffers and preservatives. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

Techniques for preparing pharmaceutical compositions are generally known in the art as exemplified by Remington's Pharmaceutical Sciences, 16th Ed. Mack Publishing Company, 1980.

Proteins extracted, isolated or produced by a method described herein according to any example can also be immobilized in or on a solid or semi-solid support. Exemplary semi-solid supports are generally matrices made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone.

Exemplary solid supports include plastics, bandages, sutures, stents, pace makers, cochlear implants or bone and/or spinal implants.

Compositions, semi-solid supports and solid supports described herein can comprise additional components. For example, the additional component is a compound (e.g., a protein) that provides a therapeutic or cosmetic benefit or that enhances a therapeutic or cosmetic benefit of the extracted protein or that interacts with or binds the extracted protein, e.g., to form a complex.

Compositions, semi-solid supports and solid supports described herein are suitable for a variety of uses, including pharmaceutical, veterinary, cosmetic, cosmaceutical or for implantation or application onto a subject. Semi-solid and solid supports are useful for bulking a tissue, correcting a tissue defect, and sealing a wound. Similarly, compositions that are tissue matrices are useful for bulking a tissue, correcting a tissue defect, and sealing a wound.

Compositions, solid-supports or semi-solid supports described herein can be administered by injection, implantation, spraying, wiping, pouring, pasting or contacting.

The present disclosure also encompasses medical devices comprising a protein isolated, extracted or produced by a method described herein. The term “medical device” encompasses any composition of matter that a regulatory agency classes as a medical device. Thus, this term can encompass, bandages and other dressings, sutures, implants, syringes, stents, needless injectors and pacemakers amongst others.

### Kits

The present disclosure also provides a kit comprising a carboxylic acid packaged with instructions for use in a method described herein.

In one example, the kit additionally comprises a microorganism for use in producing a population of microorganisms expressing a soluble protein to be extracted.

In another example, the kit comprises a microorganism for use in producing a population of microorganisms expressing a soluble protein to be extracted and instructions to extract the protein by performing a method described herein.

In another example, the kit comprises a protein extracted by performing a method of the disclosure.

In another example, the kit comprises a solid support or semi-solid support or medical device according to the present disclosure.

### EXAMPLES

The following examples demonstrate that different carboxylic acids are useful for extracting soluble protein (e.g., recombinant soluble protein) from microorganisms (e.g., E. coli).

### Example 1

### Comparison of Different Methods for Extraction of Recombinant Protein

Protocols making use of carboxylic acids, n-propanol or urea were tested for their ability to extract a recombinant protein having a pI of about 7.5 and a MW of about 72 kDa from bacterial cells.

Extraction solutions tested were:

1. 100 mM (0.57% (v/v)) acetic acid (room temperature);
2. 100 mM actetic acid (0.57% (v/v)) (at 4° C.);
3. 2.2 M (10% (w/v)) formic acid (4° C.);
4. 60% n-propanol;

### 5. 15 mM NaOH; and

6. urea extraction solution.

Table 3 shows the weight of cells treated with each solution.

TABLE 3
Treatment groups.

| Treatment | Weight of cells |
|---|---|
| 100 mM (0.57% (v/v)) acetic acid (room temperature) | 1.14 g |
| 100 mM actetic acid (0.57% (v/v)) (at 4° C.) | 1.22 g |
| 2.2M (10% (w/v)) formic acid (4° C.) | 1.32 g |
| 60% n-propanol | 1.23 g |
| 15 mM NaOH | 1.26 g |
| Urea extraction solution | 1.32 g |

Five mLs of solution was added to 1 g (wet weight) of cells and mixed for 1 hour. Solutions were then sedimented using centrifugation and supernatants recovered.

Cells treated with 15 mM NaOH were incubated for 15 min before 100 μL of 1 M borate (pH 8.0) was added to adjust the pH to about 8.0 and to make the solution to about 20 mM borate.

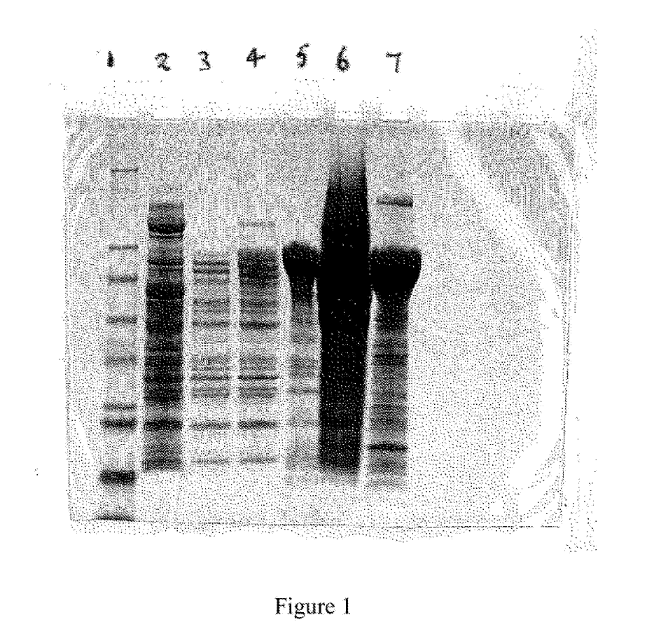

Following treatment, 10 μL of the n-propanol extraction was dried using a hetovac and resuspended in 13 μL lithium dodecyl sulphate (LDS) sample buffer for loading onto a 4-12% SDS-polyacrylamide gel electrophoresis gels. For all other samples, 10 μL of supernatant was mixed with 3 μL LDS sample buffer and loaded onto the gel. Results are depicted in FIG. 1.

The results show that NaOH or 0.57% (v/v) acetic acid did not solubilize the recombinant protein to a significant degree. The formic acid extraction dissolved the protein specifically, as did n-propanol. Urea dissolved many cellular proteins leading to an overloaded result in FIG. 1. The result with urea may also have been adversely affected by the large amounts of nucleic acid expected to be dissolved.

### Example 2

### Acetic Acid Extraction of Recombinant Protein

As demonstrated in Example 1, formic acid can be used to extract recombinant protein from E. coli. Acetic acid is similar to formic acid, in so far as it is a relatively weak carboxylic acid. Acetic acid is also inexpensive and safe to store, process and dispose of. Accordingly, acetic acid was tested for extracting the recombinant protein described in Example 1 from E. coli.

Acetic acid was tested at 2% (v/v); pH 2.71, 5% (v/v); pH 2.47 and 10% (v/v); pH 2.22.

Cells were dissoluted as follows:

2% acetic acid: 1.12 g (wet) cell paste+5 mL 2% (v/v) acetic acid.
5% acetic acid: 1.01 g (wet) cell paste+5 mL 5% (v/v) acetic acid.
10% acetic acid: 1 g (wet) cell paste+5 mL 10% (v/v) acetic acid.
Urea: 1.08 g (wet) cell paste+5 mL urea buffer.

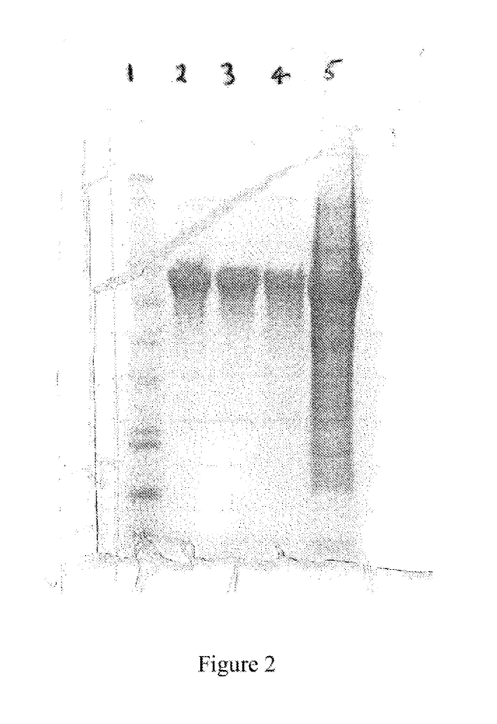

Samples were vortexed and the cell paste disrupted with a spatula to mix. Samples were then placed on a rotating mixer to mix for about 1.5 hours. One mL of sample was removed from each sample and sedimented by centrifugation at 14,000 rpm in an Eppendorf™ bench top centrifuge. Ten μL of supernatant was removed for analysis by SDS-PAGE analysis. Results are shown in FIG. 2.

SDS-PAGE analysis indicates that acetic acid dissolution is quite specific for the protein, i.e., other proteins from the cells were not solubilized to a large degree. This is in contrast to extraction with urea.

Following centrifugation, acetic extraction dissolution pelleted readily and the supernatant was slightly yellow in color. In contrast, following urea extraction and centrifugation, the supernatant was darker yellow and a soft or “slimy” pellet.

HPLC analysis indicates that the recombinant protein extracted using 2% (v/v), 5% (v/v) or 10% (v/v) acetic acid elutes at the same point on the chromatogram as control protein (i.e., produced using another method). These data indicate that the protein is correctly formed and does not appear to be damaged by the acidic extraction.

Both HPLC and SDS-PAGE analysis indicate that the 10% acetic acid extraction produced more recombinant protein than the 5% extraction, and the 5% extraction produced more than the 2% extraction. Furthermore, the acetic acid extracted protein was substantially more pure than protein extracted with urea (70.6% pure with 10% acetic acid extraction versus 48.6% pure for urea extraction as judged by HPLC analysis).

### Example 3

### Characterization of Acetic Acid Extraction Conditions

To determine the effect of acetic acid concentration, incubation time and dissolution ratio (mL acid:g wet cells) a Box-Behnken design was developed. Factors considered were:

Acetic acid concentration (% v/v): Minimum=10%, Maximum=25%
Incubation time (h): Minimum=1, Maximum=5
Dissolution ratio (mL acid/g wet cells): Minimum=1, Maximum=9
The variables were tested as set out in Table 4.

TABLE 4
Experimental design

| | Acetic acid | | Dissolution | | |
|---|---|---|---|---|---|
| Run | concentration | Time | ratio (x ml | Actual cell | Actual acid |
| order | (% v/v) | (hr) | acid:1 g cells) | mass (g) | volume (mL) |
| 1 | 10 | 1 | 5 | 1.15 | 5.7 |
| 2 | 10 | 3 | 9 | 1.05 | 9.5 |
| 3 | 17.5 | 5 | 1 | 1.09 | 1.1 |
| 4 | 10 | 3 | 1 | 1.06 | 1.1 |
| 5 | 10 | 5 | 5 | 1.05 | 5.3 |
| 6 | 17.5 | 1 | 1 | 1.08 | 1.1 |
| 7 | 25 | 5 | 5 | 1.04 | 5.2 |
| 8 | 17.5 | 3 | 5 | 1 | 5 |
| 9 | 17.5 | 3 | 5 | 1.11 | 5.6 |
| 10 | 25 | 3 | 9 | 1.06 | 9.5 |
| 11 | 17.5 | 1 | 9 | 1.06 | 9.5 |
| 12 | 25 | 3 | 1 | 1.11 | 1.1 |
| 13 | 17.5 | 3 | 5 | 1.02 | 5.1 |
| 14 | 17.5 | 5 | 9 | 1.04 | 5.2 |
| 15 | 25 | 1 | 5 | 1.07 | 5.4 |

All reactions were vortexed and any cell mass adhered to the reaction tube mixed with a glass Pasteur pipette before being placed on a rotating mixer for the reaction time.

Reaction 1 was sampled and then returned to the rotating mixer for a further 19 hours.

Reactions were vortexed before sampling to ensure complete mixing of the solution. At each time point, 1 mL of solution was removed from the reaction, centrifuged for 4 min at 14 000 rpm in an Eppendorf™ centrifuge and about 500 μL of the supernatant removed and stored before analysis by HPLC. Results are summarized in Table 5.

TABLE 5
Results of extractions

| | | | | | Total | Total | Cell | Mg | Ferment |
|---|---|---|---|---|---|---|---|---|---|
| | μg | % | | Conc | volume | protein | mass | protein/g | g/L (@130 g |
| Sample | protein | purity | vol | (mg/mL) | (ml) | (mg) | (g) | cells | cell/L) |
| Control | 10 | 93.7 | 10 | | | | | | |
| 1 | 1.9 | 41.3 | 10 | 0.2 | 6.9 | 1.3 | 1.15 | 1.1 | 0.15 |
| 2 | 1 | 40.4 | 10 | 0.1 | 10.6 | 1.1 | 1.05 | 1.1 | 0.14 |
| 3 | 5.7 | 37 | 10 | 0.6 | 2.2 | 1.2 | 1.09 | 1.1 | 0.15 |
| 4 | 4.8 | 25.8 | 10 | 0.5 | 2.2 | 1.1 | 1.06 | 1 | 0.13 |
| 5 | 1.6 | 36.3 | 10 | 0.2 | 6.4 | 1.1 | 1.05 | 1 | 0.13 |
| 6 | 5.4 | 36.6 | 10 | 0.5 | 2.2 | 1.2 | 1.08 | 1.1 | 0.14 |
| 7 | 8.8 | 75.5 | 10 | 0.9 | 6.2 | 5.5 | 1.04 | 5.2 | 0.68 |
| 8 | 3.9 | 57.5 | 10 | 0.4 | 6 | 2.3 | 1 | 2.3 | 0.3 |
| 9 | 3 | 49.2 | 10 | 0.3 | 6.7 | 2.0 | 1.11 | 1.8 | 0.23 |
| 10 | 8.2 | 77.8 | 10 | 0.8 | 10.6 | 8.7 | 1.06 | 8.2 | 1.07 |
| 11 | 2.5 | 66.4 | 10 | 0.2 | 10.6 | 2.6 | 1.06 | 2.5 | 0.32 |
| 12 | 4.2 | 28 | 10 | 0.4 | 2.2 | .9 | 1.11 | 0.8 | 0.11 |
| 13 | 2.9 | 45.6 | 10 | 0.3 | 6.1 | 1.7 | 1.02 | 1.7 | 0.22 |
| 14 | 3.5 | 52 | 10 | 0.3 | 6.2 | 2.1 | 1.04 | 2.1 | 0.27 |
| 15 | 5.8 | 58.9 | 10 | 0.6 | 6.5 | 3.8 | 1.07 | 3.5 | 0.46 |
| 1 at 20 | 2.2 | 43.2 | 10 | 0.2 | 6.9 | 1.6 | 1.15 | 1.3 | 0.18 |
| hours | | | | | | | | | |

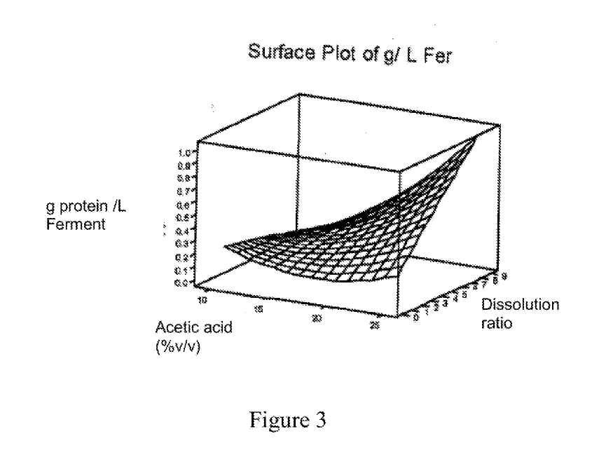

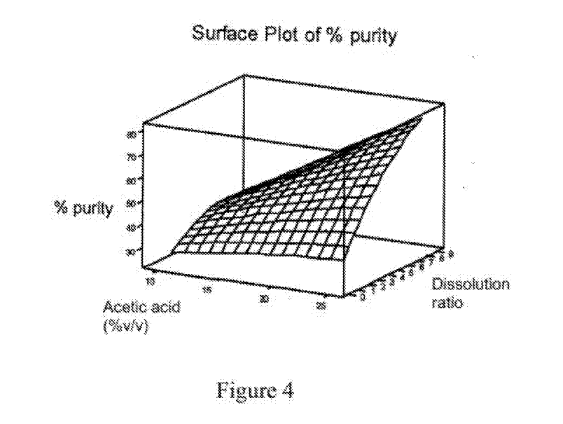

Results are also depicted in FIGS. 3 and 4.

The results show that acetic acid was capable of extracting recombinant protein following incubations of various lengths of time and that the time of incubation did not substantially affect recovery or purity. Thus, these results demonstrate that lysis of cells and/or protein extraction under the conditions tested occurs rapidly, i.e., the level of lysis and/or extraction is similar at one hour to the level at 3, 5 or 20 hours. This appears counter-intuitive since it would be expected that longer incubation times would result in a higher level of cell lysis. The results also demonstrate that acetic acid concentration and dissolution ratio had the strongest affects on yield (concentration: estimated regression co-efficient 0.22; p<0.005; ratio: estimated regression co-efficient 0.16, p<0.05) and purity (concentration: estimated regression co-efficient 12.05; p<0.005; ratio: estimated regression co-efficient 13.65, p<0.005). Both purity and yield increased with increasing concentration and dissolution ratio.

### Example 4

### Effect of Amount of Acetic Acid on Protein Recovery and Purity

Assays were performed to determine the effect of various concentrations of acetic acid and the effect of extraction time on the amount of recombinant protein and purity of protein extracted from E. coli. Cells expressing the same protein as in Example 1 were assayed.

Extraction (using acetic acid concentrations (%): 25, 37.5, 50, 62.5, 75, 87.5, 100) were left to go to completion (i.e. overnight extraction for 20±4 hrs). The supernatant was then analysed by RP-HPLC and the amount of recombinant protein extracted and the purity produced determined by comparison to the peak area and purity of a reference standard.

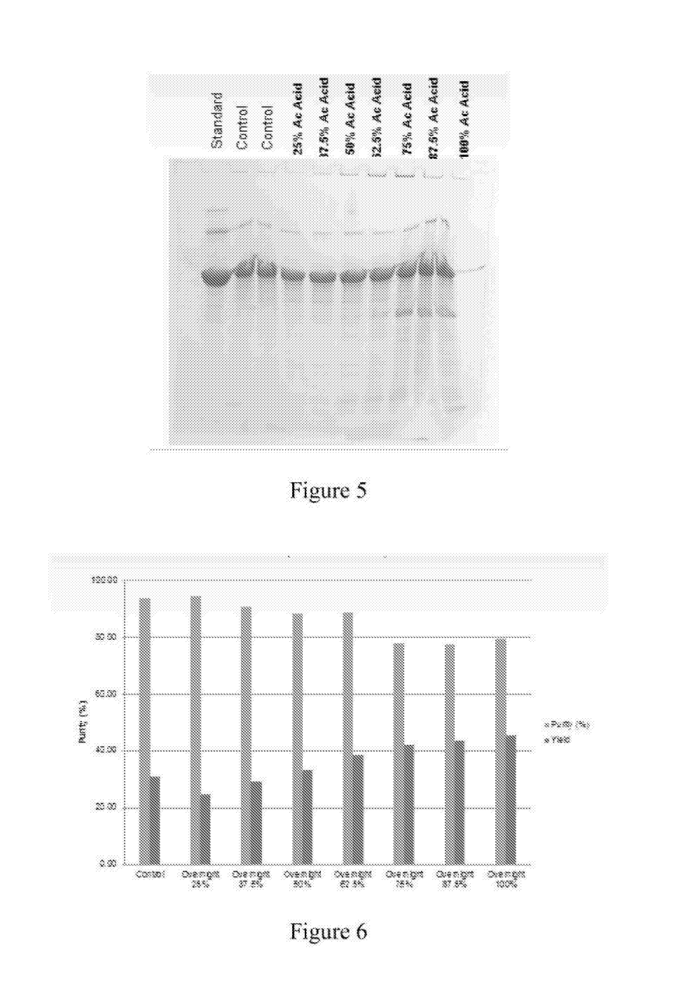

FIG. 5 shows SDS-PAGE analysis demonstrating that increasing concentrations of acetic acid extracted increased levels of recombinant protein, however increased levels of contaminants were also extracted.

Results of analysis of the extracts using RP-HPLC are shown in Table 6. These data indicate that purity begins to decrease above about 62.5%.

TABLE 6
Effect of acetic acid concentration on the extraction of recombinant protein
from E. coli cells following overnight incubation as analysed by RP-HPLC

| Acetic acid | Purity | Yield |
|---|---|---|
| (%) | (%) | (g/L) |
| 25% Control | 93.54 | 1.54 |
| 25% | 94.45 | 1.24 |
| 37.5% | 90.56 | 1.47 |
| 50% | 88.28 | 1.67 |
| 62.5% | 88.57 | 1.93 |
| 75% | 78.11 | 2.11 |
| 87.5% | 77.67 | 2.19 |
| 100% | 79.40 | 2.28 |

The data set out in Table 6 are also presented graphically in FIG. 6, which shows a correlation between acetic acid concentration and yield of recombinant protein and an inverse correlation between acetic acid concentration and purity once a threshold concentration has been reached.

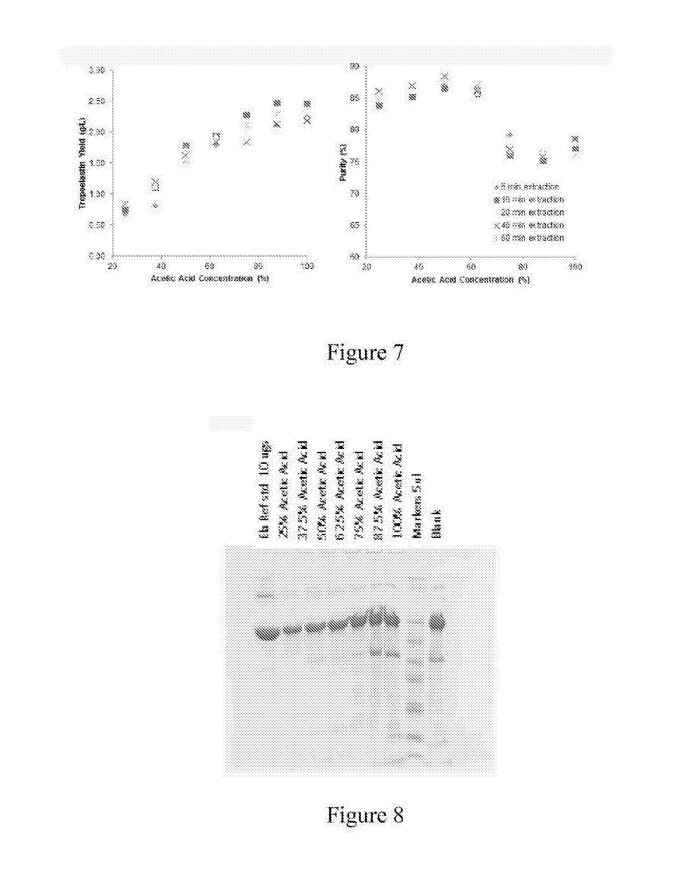

One hundred μL of the reactions prepared as described above were also sampled at a variety of timepoints (5, 10, 20, 40 and 60 minutes), centrifuged immediately and the supernatant removed. The supernatant was analysed by RP-HPLC and compared to a reference standard to determine quantity and purity. Results of this analysis are presented in Tables 7 and 8 and FIG. 7. SDS-PAGE analysis was also performed to provide an orthogonal method of comparing the purity of the protein produced and results are depicted in FIG. 8.

TABLE 7
Effect of acetic acid concentration on the extraction of recombinant protein
from E. coli cells following 5, 10, 20, 40 and 60 minute incubations

| | Acetic acid concentration | | | | | | |
|---|---|---|---|---|---|---|---|
| | 25% | 37.50% | 50% | 62.50% | 75% | 87.50% | 100% |
| min | Protein extracted (g/L) | | | | | | |
| 5 | 0.68 | 0.82 | 1.52 | 1.80 | 2.27 | 2.11 | 2.25 |
| 10 | 0.75 | 1.11 | 1.79 | 1.95 | 2.28 | 2.48 | 2.47 |
| 20 | 0.86 | 1.14 | 1.51 | 1.94 | 1.97 | 2.05 | 2.22 |
| 40 | 0.85 | 1.21 | 1.63 | 1.84 | 1.84 | 2.13 | 2.19 |
| 60 | 0.86 | 1.18 | 1.74 | 1.86 | 2.12 | 2.29 | 2.39 |

TABLE 8
Effect of acetic acid concentration on the purity of recombinant protein
extracted from E. coli cells following 5, 10, 20, 40 and 60 minute
incubations

| | Acetic acid concentration | | | | | | |
|---|---|---|---|---|---|---|---|
| | 25% | 37.50% | 50% | 62.50% | 75% | 87.50% | 100% |
| min | Protein purity (%) | | | | | | |
| 5 | 84.6 | 85.0 | 87.0 | 86.8 | 79.2 | 76.6 | 78.5 |
| 10 | 83.8 | 85.3 | 86.5 | 85.7 | 76.1 | 75.3 | 77.3 |
| 20 | 84.9 | 87.3 | 88.1 | 85.9 | 77.8 | 76.7 | 78.0 |
| 40 | 86.1 | 87.0 | 88.3 | 86.1 | 77.0 | 75.9 | 78.6 |
| 60 | 84.8 | 86.7 | 88.2 | 87.2 | 76.4 | 75.6 | 76.2 |

### Example 5

### Extraction of a Fusion Protein Using Acetic Acid

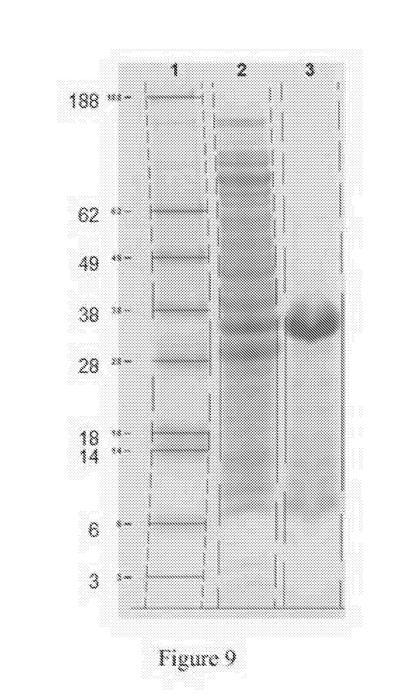

Bacterial cells expressing a fusion protein comprising three copies of a peptide and having a pI of about 4.6 were grown, harvested and frozen. Cells were then lysed or the fusion protein was extracted by contacting the cells with a solution of 25% acetic acid (v/v) for one hour, centrifuged and the supernatant collected. SDS-PAGE analysis of the resulting compositions is shown in FIG. 9. These data demonstrate that extraction with acetic acid substantially reduced contamination by host cell proteins. These data also show that the extraction method described herein is applicable to fusion proteins and/or to proteins having acidic pIs.

1. A method for extracting a soluble protein from a population of microorganisms, the method comprising contacting the population of microorganisms expressing the soluble protein with an amount of a solution comprising from about 1% (v/v) to less than 50% (v/v) carboxylic acid effective to extract the soluble protein from the population of microorganisms.

2. The method of claim 1, wherein the solution comprises from about 1% (v/v) to about 40% (v/v) carboxylic acid.

3. The method of claim 1, wherein the solution comprises from about 25% (v/v) to about 37.5% (v/v) carboxylic acid.

4. The method of any one of claims 1 to 3, wherein the carboxylic acid is added at a dissolution ratio from about 1:1 (milliliters of acid:grams of microorganism (wet)) to about 20:1 (milliliters of acid:grams of microorganism (wet)).

5. The method of any one of claims 1 to 3, wherein the carboxylic acid is added at a dissolution ratio from about 5:1 (milliliters of acid:grams of microorganism (wet)) to about 10:1 (milliliters of acid:grams of microorganism (wet)).

6. The method of any one of claims 1 to 5, wherein the carboxylic acid has a pKa of at least about 3.

7. The method of any one of claims 1 to 6, wherein the carboxylic acid is acetic acid or formic acid.

8. The method of any one of claims 1 to 6, wherein the carboxylic acid is acetic acid.

9. The method of any one of claims 1 to 8, comprising contacting the population of microorganisms with the solution for about 5 hours or less.

10. The method of any one of claims 1 to 8, comprising contacting the population of microorganisms with the solution for about 1 hour or less.

11. The method of any one of claims 1 to 10 additionally contacting the population of microorganisms with a detergent.

12. The method of any one of claims 1 to 11, wherein the soluble protein is recombinantly expressed by the population of microorganisms.

13. The method of any one of claims 1 to 12, wherein the population of microorganisms is bacteria, yeast or fungi.

14. The method of any one of claims 1 to 12, wherein the population of microorganisms is bacteria.

15. The method of any one of claims 1 to 12, wherein the population of microorganisms is Gram negative bacteria.

16. The method of any one of claims 1 to 12, wherein the population of microorganisms is Escherichia coli.

17. The method of any one of claims 1 to 16, wherein the soluble protein has a pI that permits the protein to remain soluble in the solution when the protein is extracted from the population of microorganisms.

18. The method of any one of claims 1 to 17, wherein the soluble protein has a pI of 7.5 or greater.

19. The method of any one of claims 1 to 17, wherein the soluble protein has a pI of 6 or less.

20. The method of any one of claims 1 to 19, wherein the protein is selected from the group consisting of a scleroprotein, a naturetic peptide, a chaperonin, a heat shock protein, an interleukin, an interferon, a fusion protein and a protein comprising a variable domain of an antibody.

21. A method for purifying a soluble protein from a population of microorganisms, the method comprising performing the method of any one of claims 1 to 20 and purifying the soluble protein extracted by the method.

22. The method of claim 21 additionally comprising admixing the soluble protein with another compound.

23. The method of any one of claims 1 to 22 additionally comprising modifying the purified or extracted protein.

24. A method for producing a modified protein, the method comprising obtaining the protein purified by the method of any one of claims 21 to 23 and modifying the protein or obtaining a protein extracted by the method of any one of claims 1 to 20 and modifying the protein.

25. The method of claim 23 or 24, wherein modifying the protein comprises conjugating another compound to the protein.

26. The method of any one of claims 21 to 25 additionally comprising formulating the protein into a composition.

27. The method of any one of claims 21 to 25 additionally comprising immobilizing the protein onto or into a solid support or semi-solid support.

28. A method for producing a composition, the method comprising obtaining the protein extracted, purified or produced by the method of any one of claims 1 to 25 and formulating the protein into a composition.

29. A method for producing a solid or semi-solid support, the method comprising obtaining the protein extracted, purified or produced by the method of any one of claims 1 to 25 and immobilizing the protein onto or into a solid support or semi-solid support.

30. The method of any one of claims 26 to 29, wherein the composition, solid support or semi-solid support is for administration or application to or on a human or a non-human animal.

31. The method of any one of claims 26 to 31, wherein the composition, solid support or semi-solid support is for pharmaceutical use, cosmetic use, cosmaceutical use, veterinary use or for implantation.

32. A protein, composition, solid support or semi-solid support produced by performing a process comprising performing the method of any one of claims 1 to 31.

33. The protein, composition, solid support or semi-solid support of claim 32 for use in medicine.

34. The protein, composition, solid support or semi-solid support of claim 32, which is for pharmaceutical use, cosmetic use, cosmaceutical use, veterinary use or for implantation.

35. A method of treating a condition, the method comprising administering to a subject in need thereof the protein, composition, solid support or semi-solid support of claim 32.

36. Use of the protein, composition, solid support or semi-solid support of claim 32 in the manufacture of a medicament for treating a condition.

37. A device comprising the protein, composition, solid support or semi-solid support of claim 32.

38. The device of claim 35, which is a syringe, a patch or an implant.
